(19) United States
(12) Patent Application Publication
(10) Pub. No.: US 2013/0095474 A1
(43) Pub. Date: Apr. 18, 2013

(54) DESIGN OF STEM-LOOP PROBES AND UTILIZATION IN SNP GENOTYPING

(71) Applicants: JOSEPH MAMONE, Hillsborough, NJ (US); Howard Gamper, Philadelphia, PA (US)

(72) Inventors: JOSEPH MAMONE, Hillsborough, NJ (US); Howard Gamper, Philadelphia, PA (US)

(21) Appl. No.: 13/272,961

(22) Filed: Oct. 13, 2011

(51) Int. Cl. C12Q 1/68; G01N 21/64; C07H 21/04

(52) U.S. Cl. 435/6.11; 536/24.3

(57) ABSTRACT

Stem-loop probe for single nucleotide polymorphism (SNP) genotyping of individual SNP nucleic acid target sequences has first, second, and third single stranded nucleic acid portions. Second portion is between the first and third portions. First and third portions build a double stranded, intramolecular stem. Second portion forms a single stranded oligonucleotide loop with a nucleotide sequence, complementary to individual SNP nucleic acid target sequences. The nucleotide sequence of probe is matches probe/target hybrids have a melting point Tₘ that is at least 5° C. higher than Tₘ of mismatched probe/target hybrids. A method of detecting SNP in nucleic acid containing samples uses a pair of stem-loop probes for SNP genotyping of two individual SNP nucleic acid target sequences. The probes comprise the same first, second, and third portions and a ratio of perfect match probe/target hybrids to mismatched probe/target hybrids is detected at a certain temperature.

### FIELD OF TECHNOLOGY

The present invention relates to stem-loop probes for single nucleotide polymorphism (SNP) genotyping of individual SNP nucleic acid target sequences. The stem-loop probes comprise first, second, and third single stranded nucleic acid portions. The second single stranded nucleic acid portion is located between the first and the third single stranded nucleic acid portions. The first and the third single stranded nucleic acid portions build a double stranded, intramolecular stem. The second single stranded nucleic acid portion forms a single stranded oligonucleotide loop with a nucleotide sequence that is complementary to individual SNP nucleic acid target sequences. The present invention also relates to a method of detecting single nucleotide polymorphism (SNP) in nucleic acid containing samples, utilizing a pair of stem-loop probes for SNP genotyping of two individual SNP nucleic acid target sequences of a sample.

### RELATED PRIOR ART

In particular in forensic medicine, the analysis of DNA (deoxyribonucleic acid) is routinely used, e.g. for identifying human individuals or for profiling a group of individuals. Various methods have been established for different purposes, the most frequently used tool involves the analysis of STR (short tandem repeats). However, in the last years, the interest in the use of SNPs (single nucleotide polymorphism) has increased. Such SNPs are single-based variations at a unique physical location of the genomic DNA of an individual, and are currently considered to be the most common class of human polymorphism (with an estimated occurrence of 1 in every 1000 bases in the human genome). A number of SNPs are known to be associated with distinct diseases, e.g. when being located in the coding region of a gene. Furthermore, there are other SNPs known to be connected to specific populations, e.g. with US Caucasians or Hispanics, which is particularly useful in forensic medicine (taken from “SNP Typing in Forensic Genetics”, B. Sobrino and A. Carracedo, Methods in Molecular Biology 2005, Vol. 297: 107-126).

The use of SNPs offers several advantages over the analysis of STRs:
SNPs have lower mutation rates than STRs, which increases the reliability of a population analysis.
SNPs may be analyzed from short amplicons, which is desirable in particular when using e.g. degraded samples.
SNPs are suitable for high-throughput techniques and automated processing because SNP assays are all simpler than current STR assays. For example, single base extension is very simple. STR assays currently involve sizing fragments via capillary electrophoresis (CE), which is not automated.
Current STRs only occur in non-coding regions and can only be selected from such non-coding regions. SNPs can be found either in non-coding (which is preferred for generic human identification where phenotypic traits are not included) or can be found in coding regions linked to phenotypic information (e.g. eye color). The SNPs that are preferred according to the present invention are selected from non-coding regions.

Generally, four major assay principles of SNP genotyping are known: allele specific hybridization, primer extension, oligonucleotide ligation, and invasive cleavage. Allele specific hybridization involves the generation of two allele-specific hybridization probes specific for the nucleotide polymorphism found in the analyzed SNP. Only the hybridization of probe and SNP region with a perfect nucleotide match results in stable hybrids, while the hybrid with a one-base mismatch is unstable at the same temperature. Known methods of detecting stable and unstable hybrids are e.g. FRET (Fluorescence resonance energy transfer) and Array hybridization.

Array hybridization for genotyping SNPs in human is for example known from the patent document U.S. Pat. No. 7,361,468 B2. Here, short oligonucleotides including both allele specific polymorphism probes are spotted in a microarray. An advantage of this array hybridization is that many SNPs may be analyzed in parallel. However, the design of the probes when analyzing different SNPs in parallel may raise some problems, as the efficiency of hybridization and the stability of hybrids is not only based on the polymorphic site but also on the SNP flanking sequence. This in turn affects the melting temperature of the resulting hybrids. According to this patent document, the use of a multitude of immobilized probes for each SNP, with each probe differing in the respective sequences of the flanking sites may solve this problem.

### OBJECTS AND SUMMARY OF THE PRESENT INVENTION

It is an object of the present invention to suggest a collection of stem-loop probes that act simultaneously in a single sample and in the same temperature range for assessing multiple SNP sites in a single sample.

It is a further object of the present invention to suggest a method of detecting multiple SNP sites in a single sample using a collection of stem-loop probes that act simultaneously in said single sample and in the same temperature range.

A first objective is achieved by a stem-loop probe for single nucleotide polymorphism (SNP) genotyping of individual SNP nucleic acid target sequences. The stem-loop probe comprises first, second, and third single stranded nucleic acid portions. The second single stranded nucleic acid portion is located between the first and the third single stranded nucleic acid portions. The first and the third single stranded nucleic acid portions building a double stranded, intramolecular stem and the second single stranded nucleic acid portion forms a single stranded oligonucleotide loop with a nucleotide sequence that is complementary to individual SNP nucleic acid target sequences. The stem-loop probe according to the present invention is characterized in that the nucleotide sequence of the stem-loop probe is chosen such that perfect match probe/target hybrids have a melting point Tₘ that is at least 5° C. higher than the Tₘ of mismatched probe/target hybrids.

A second objective is achieved by proposing a method of detecting single nucleotide polymorphism (SNP) in nucleic acid containing samples, utilizing a pair of stem-loop probes for SNP genotyping of two individual SNP nucleic acid target sequences of a sample. The stem-loop probes comprise first, second, and third single stranded nucleic acid portions. The second single stranded nucleic acid portion is located between the first and the third single stranded nucleic acid portions.

The first and the third single stranded nucleic acid portions build a double stranded, intramolecular stem and the second single stranded nucleic acid portion forms a single stranded oligonucleotide loop with a nucleotide sequence that is complementary to one of the individual SNP nucleic acid target sequences. The method of detecting SNP in nucleic acid containing samples according to the present invention is characterized in that a ratio of perfect match probe/target hybrids to mismatched probe/target hybrids is detected at a certain temperature, and in that the nucleotide sequence of the stem-loop probe is chosen such that the perfect match probe/target hybrids having a melting point Tₘ that is at least 5° C. higher than the Tₘ of mismatched probe/target hybrids.

Additional features and preferred embodiments of the present invention derive from the depending claims in each case.

### ADVANTAGES OF THE INVENTION

Known techniques (see e.g. U.S. Pat. No. 7,361,468 B2 and U.S. Pat. No. 7,582,421 B2) utilize arrays of capture probes over which a DNA sample is run. In contrast to this prior art and with the present invention, the DNA sample are captured and probes are run over the DNA samples. This provides the particular advantage that there is no need to utilize specially prepared microarrays.

### BRIEF INTRODUCTION OF THE DRAWINGS

The enclosed drawings are used to explain the present invention and shall not limit its scope. It is shown in:

FIG. 1 a graph illustrating the signal ratio Cy5/FAM of investigated alleles on a semi-logarithmic scale;

FIG. 2 the results of a first series of exemplary hybridization experiments, the discrimination ratio being defined as the signal of perfect match probe/target hybrid divided by the signal of mismatched probe/target hybrid for the same probe, wherein:

FIG. 2A shows the result of a first experiment, revealing the A-T perfect match signal of a first probe;

FIG. 2B shows the result of a second experiment, revealing the C-T mismatch signal of the same first probe;

FIG. 2C shows the result of a third experiment, revealing the A-G mismatch signal of a second probe; and

FIG. 2D shows the result of a fourth experiment, revealing the C-G perfect match signal of the same second probe.

FIG. 3 the results of a second series of exemplary hybridization experiments, the specificity being defined as the signal of perfect match probe/target hybrid divided by the signal of mismatched probe/target hybrid for the same target, wherein:

FIG. 3A shows the result of a first experiment, revealing the A-T perfect match signal of a first probe with a first target;

FIG. 3B shows the result of a second experiment, revealing the A-G mismatch signal of a second probe with the same first target;

FIG. 3C shows the result of a third experiment, revealing the C-T mismatch signal of the first probe with a second target; and

FIG. 3D shows the result of a fourth experiment, revealing the C-G perfect match signal of the second probe with the same second target;

FIG. 4 the results of a third series of exemplary hybridization experiments, the resolving power being defined as the fold change in FAM/Cy5 or Cy5/FAM signal between a homozygous and a heterozygous target, wherein;

FIG. 4A shows the result of a first experiment, revealing the A-T perfect match signal of a first probe with a first target and the A-G mismatch signal of a second probe with the same first target;

FIG. 4B shows the result of a second experiment, revealing the A-T perfect match signal of the first probe with the first target and the C-G perfect match signal of the second probe with the second target; and

FIG. 4C sows the result of a third experiment revealing the C-T mismatch signal of the first probe with the second target and the C-G perfect match signal of the second probe with the second target;

FIG. 5 discrimination of Amelogenin Intron 1 X and Y alleles and demonstration of use of Tₘ data to design probes, wherein:

FIG. 5A shows a comparison of melting curves of C224-B (X-probe) hybridized with X- and Y-alleles of Amelogenin Intron 1;

FIG. 5B shows a comparison of melting curves of T224-B (Y-probe) hybridized with Y- and X-alleles of Amelogenin Intron 1;

FIG. 5C shows the first derivatives of the graph of FIG. 5A; and

FIG. 5D shows the first derivatives of the graph of FIG. 5B;

FIG. 6 data from differential hybridization of immobilized female (7437) or male (7432) Amelogenin amplicons, an X/Y typing assay using data from FIG. 5 at a single temperature, wherein:

FIG. 6A shows interrogation of a DNA sample derived from a female contributor (XX or homozygous for the X allele), and

FIG. 6B shows interrogation of a DNA sample derived from a male contributor (XY or heterozygous for the X and Y alleles);

FIG. 7 an overview over SNPs identified so far and a listing of the respective melting temperatures Tₘ;

FIG. 8 graphic models of stem-loop probes, wherein:

FIG. 8A shows the basic parts of a stem-loop probe; and

FIG. 8B shows a typical sequence of a stem-loop with a 17 nucleotide loop.

### DETAILED DESCRIPTION OF THE PRESENT INVENTION

The present invention relates to the detection of single nucleotide polymorphisms (SNPs) for profiling a group of individuals or for detecting individuals in particular in forensic medicine. The following is an explanation of preferred embodiments of the present invention and shall not limit the gist and scope of this invention.

Design of Stem-Loop Probes:

One of the main ideas and targets of the present invention is the design of a collection of stem-loop probes that can act simultaneously in a single sample and in the same temperature range for assessing multiple SNP sites in a single sample. More particularly, the whole concept is based on how carefully designed the stem-loop probe is in order to have exactly the correct Tₘ to prefer to form hybrids with matching templates only providing significant discrimination between two very similar sequences that may differ in one nucleotide only.

As a first test model, genotyping of SNP 9 in a representative human genomic DNA sample was chosen. SNP 9 is a biallelic Guanine/Cytosine (G/C) single nucleotide polymorphism (SNP) found in human DNA on chromosome 9 (NCBI identifier rs763869). The inventors of the current invention decided utilizing two stem-loop probes to genotype this SNP since such probes are more discriminating than simple linear probes.

The sequence and length of the stem determines how stable the stem-loop structure is relative to the binding strength of the probe with the target (i.e. as a template duplex structure). Longer sequences make generally stronger stems, higher G-C base pairs make generally stronger stems, but it is well known that the strength of the duplex is sequence context dependent (not just a function of the number of G-C base pairs). According to such consideration, the inventors decided to build a stem with 5 base pairs (see guideline No. 1, below).

The loop is what hybridizes to the SNP region, so the SNP and immediate flanking regions dictate the loop sequence. There could be longer (more tightly binding, higher Tₘ) loops or shorter (less tightly binding, lower Tₘ) loops. There could also be non-sense bases at either end of the loop region to make the stem-loop more or less stable and drive the equilibrium either direction. More specifically, the length should not be so long that there is non-specific binding at the hybridization temperature. The experiment to determine these is to examine the background measurements at the desired hybridization temperature. If too long, the mismatch will be more stable and high background will result. According to such consideration, the inventors decided to build a stem-loop with 15-18 nucleotides (see guideline No. 2, below).

During expected hybridization, the double-stranded stem opens, but does not bind to target DNA. There should be no influence of “flanking sequences” in the target sequence, i.e. the target sequence should be free of significant secondary structure at the hybridization temperature, because if the flanking sequences form a somewhat stable structure in the target without the probe present, this would make probe binding less efficient, requiring a stronger binding probe. Also, the immediately adjacent flanking nucleotides (one away from the ends of the nascent duplex) will influence binding energy of the probe in a sequence dependent way. Lastly, if there are other ways the probe can bind to the flanking region, this will effectively compete with the desired duplex. The combination of length and sequence of the loop together with the length and sequence of the stem determine the melting temperature in the hybridization reaction by affecting the equilibrium between stem-loop structure and probe-template duplex structure.

It further has been considered by the inventors to NOT have a G adjacent to the fluorescent label in the probe sequence as the Guanine will quench the fluorescence signal (see guideline No. 5, below and FIG. 8B).

The two probes, each complementary to one of the two SNP sequences, have been designed using free software available on the IDT website and according to the following guidelines:
(1) Each probe has a 5 base pair long stem in which 4 of the base pairs were

Guanine-Cytosine (G-C) (see FIG. 8B).
(2) The single-stranded loop of each probe is 15-18 nucleotides long and free of secondary structure (see FIG. 8B). Each loop is complementary to one of the two SNP sequences and is oriented symmetrically with respect to the SNP. The two probes hybridize to the same strand of the target DNA and form hybrids that have a minimal G/C content of 35-45%.
(3) The intramolecular stem of each probe and the two perfect-match probe-target hybrids have melting temperatures (Tₘ) close to 55° C. in HEPES buffer with 50 mM NaCl and 1 mM MgCl₂ in the presence of 0.4 μM probe.
(4) The Tₘ of the mismatched probe-target hybrid is depressed by at least 5° C. relative to the perfect-match hybrid.
(5) Each probe has a different fluorophore which is conjugated to the 3′ or 5′ end of the oligomer next to an Adenine (A), Thymine (T), or Cytosine (C, see e.g. FIG. 8B). The two probes designed according to the above specifications are listed below with the SNP bases underlined.

According to the above guidelines and due to some empirical determination and with some smart design ideas, the inventors arrived with the stem-loop design according to which the sequence listing attached to this patent application has been prepared. For illustration purposes, one pair of probes is displayed here:

SEQ ID: NO 56:
5′-GCGTG-GTTTTATTGCTGTCCCAGT-CACGC-FAM
SEQ ID: NO 81:
5′-GCGTG-GTTTTATTCCTGTCCCAGT-CACGC-Cy5

It is thus preferred that, when starting at the 5′ ends:
a first (1) single stranded nucleic acid portion has the SEQ ID: NO 1: GCGTG (see FIG. 8B and sequence listing).
a second (2) single stranded nucleic acid portion that is located between the first (1) and a third (3) single stranded nucleic acid portions has e.g. the SEQ ID: NO 2: GTTTTATTGCTGTCCCAGT (compare with FIG. 8B and sequence listing); and
the third (3) single stranded nucleic acid portion has the SEQ ID: NO 3: CACGC (see FIG. 8B and sequence listing).
a fourth (4) single stranded nucleic acid portion that is located between a first (1) and a third (3) single stranded nucleic acid portions has the SEQ ID : NO 4: GTTTTATTCCTGTCCCAGT (see sequence listing).

Accordingly, the preferred full length oligonucleotide comprising the 1^{st}, 2^{nd}, and 3^{rd} nucleic acid portion has the SEQ ID: NO 56 and the preferred full length oligonucleotide comprising the 1^{st}, 4^{th}, and 3^{rd} nucleic acid portion has the SEQ ID: NO 81. A number of 109 preferred full length oligonucleotides with conjugated fluorophors (comprising the full length oligonucleotides with the SEQ ID: NOs 56 and 81 above) are listed in the attached sequence listing.

The chosen fluorophors FAM, and/or Cy5, and/or Q670 are known in the art and exhibit the following characteristics:
FAM: Excitation at wavelength of (absorption maximum at) 485 nm and emission (emission maximum) at 520 nm
Cy5: Excitation at wavelength of (absorption maximum at) 633 nm and emission (emission maximum) at 666 nm
Q670: Excitation at wavelength of (absorption maximum at) 644 nm and emission (emission maximum) at 670 nm.

These two probes have been found to perfectly match the target DNA of SNP 9 locus, either with or without polymorphism. In order to demonstrate this, the following experiments have been carried out:

Amplification of SNP Sequence from Genomic CEPH DNA:

PCR amplification of SNP 9 from CEPH genomic DNA 6984 (purchased from Coriell Institute, Camden, N.J., USA) was carried out in a total volume of 100 μl prepared by mixing together 63.2 μl water, 20 μl X5 HF buffer (NEW ENGLAND BIOLABS), 5 μl 10 μM primer M9F (5′-AAGTGATGGAGTTA-GGAAAAGAACC), 5 μl 10 μM primer M9R (Biotin-5′-AAGACATTAGGTGGATTC-ATAGCTG), 0.8 μl 25 mM dNTPs, 1.0 μl Phusion DNA polymerase (NEW ENGLAND BIOLABS), and 5 μl of 30 ng/μl CEPH DNA.

Additions were carried out using pipette tips with an aerosol barrier. Amplification was conducted in a thermocycler programmed for 2 min at 98° C. (hot start), 35 cycles of 30 sec at 98° C. (denaturation step), 1 min at 60° C. (annealing step), and 15 sec at 72° C. (extension step) followed by 1 min at 72° C. and storage at 4° C. A 5 μl aliquot of the PCR reaction was analyzed by electrophoresis in a 2% agarose gel in X1 TAE for purity (single band 95 by in length) and yield (40 pmoles of product in the 100 μl PCR reaction). One strand of the PCR product was biotinylated at the 5′ end.

Immobilization of PCR Product on Streptavidin-Coated Magnetic Beads:

A volume of 300 μl of a 10 mg/ml suspension of streptavidin-coated magnetic beads (Dynabeads M-280 Streptavidin; INVITROGEN) was transferred to a 0.6 ml microcentrifuge tube. The beads were then washed 3 times with 300 μl of BW buffer (50 mM Tris-HCl pH 7.5, 0.5 mM EDTA, 1 M NaCl) using a magnetic stand (INVITROGEN) to pellet the beads between washes. After the third wash the beads were resuspended in 150 μl 2× BW buffer and 25 μl aliquots (containing 0.5 mg beads) were dispensed into five 1.5 ml microcentrifuge tubes. To four of the tubes was added 25 μl of 2× BW buffer followed by either 1 μl of 80 μM T39, 1 μl of 80 μM T40, 1 μl of 40 μM T39+1 μl 40 of μM T40, or 1 μl water a (no-target control). Additional water was added to each of these tubes to give a final volume of 100 μl.

T39 and T40 (shown in order below) are single-stranded biotinylated 60-mer oligonucleotides that replicate the two SNP sequences and are used as standards for calibrating the hybridization readout. To a fifth tube was added 25 μl of bead suspension, 75 μl of 2× BW buffer, and 100 μl of PCR reaction mixture. Binding of biotinylated standards or PCR product+excess biotinylated primer to the streptavidin-coated magnetic beads took place at room temperature for 30 min. After pelleting the beads on a magnetic stand, the supernatants were removed. Beads loaded with synthetic single-stranded targets were rinsed 3 times with 100 μl aliquots of BW buffer and 3 times with 100 μl aliquots of HD buffer. After resuspension in 100 μl of HD buffer (10 mM HEPES pH 7.5, 50 mM NaCl, 1 mM MgCl₂), the solutions were transferred to 0.5 ml microcentrifuge tubes. Beads loaded with PCR product were processed separately as described in the next step.

T39:
(SEQ ID: NO 121)
Biotin-T9-TGCTTATGAAAAATCACTGGGACAGGAATAAAACACCTGGGTTCTTTTCCT
T40:
(SEQ ID: NO 122)
Biotin-T9-TGCTTATGAAAAATCACTGGGACAGCAATAAAACACCTGGGTTCTTTTCCT

Alkaline Stripping of Non-Biotinylated PCR Strand:

Beads loaded with biotinylated PCR product were washed 3 times with 100 μl aliquots of BW buffer followed by 1 wash with 100 μl of 2× BW buffer. The beads were resuspended in 100 μl of 0.1 M NaOH and incubated 5 min at room temperature. After removal of the supernatant, the beads were washed once with 100 μl of fresh 0.1 M NaOH, 3 times with 100 μl aliquots of BW buffer, and 3 times with 100 μl aliquots of HD buffer. Pelleted beads were finally resuspended in 100 μl of HD buffer and transferred to a 0.5 ml microcentrifuge tube for hybridization.

Hybridization:

A volume of 2 μl of probe cocktail (80 μM P52 and 80 μM P77) was added to each 100 μl suspension of target bearing bead suspension and incubated at 49° C. for 20 min. The supernatant solution was removed and the beads were incubated in 100 μl of HD buffer at 49° C. for 15 min. Finally, the hybridized probe was recovered by incubating the bead suspension in 100 μl of HD buffer at 65° C. for 15 min. The supernatants were collected and transferred to a microtiter plate for reading of fluorescence.

Signal Readout and Analysis:

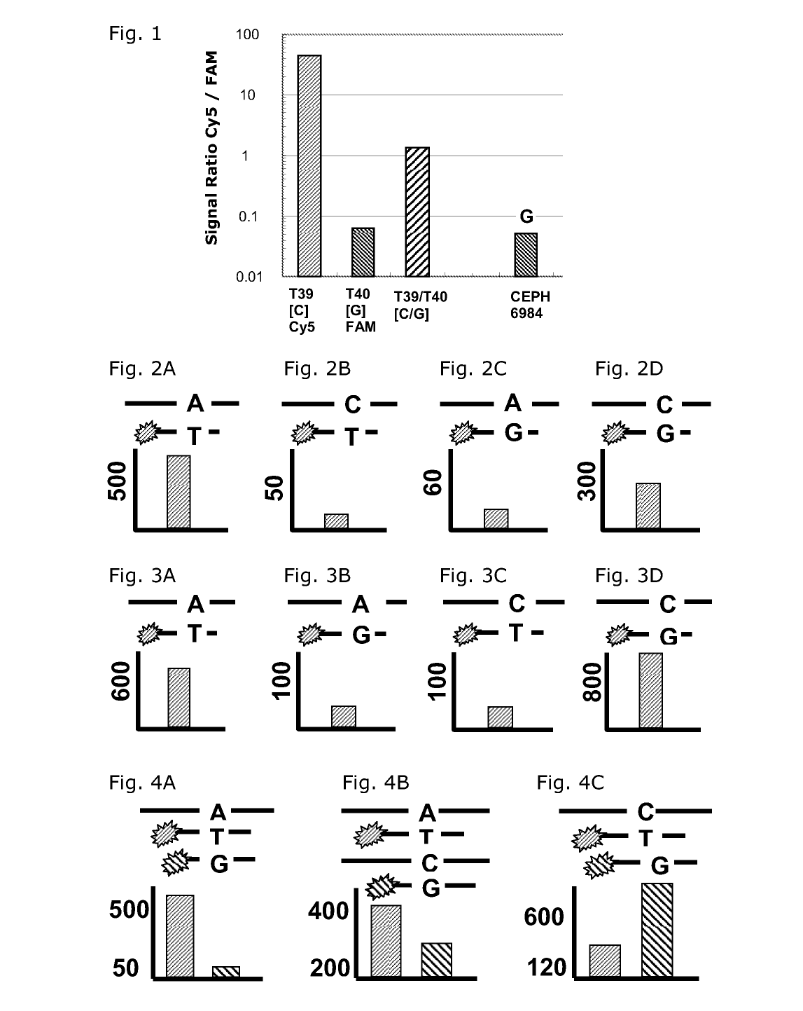

Each supernatant together with a buffer blank and a reference standard (100 μl HD buffer containing 80 pmols (pico mols) each of P52 and P77) were read for fluorescence of FAM (485 nm excitation & 520 nm emission) and Cy5 (633 nm excitation and 666 nm emission). After correcting readings for background fluorescence, the ratio of Cy5 to FAM fluorescence was used to type the CEPH 6984 DNA sample for SNP 9 as shown in FIG. 1. Allelic sequence differences—based on a specific SNP—are known for each SNP used. All stem-loop probes of one SNP-specific probe set are designed to have a hybridization optimum (=maximum) at the same temperature. In this way, the ratio between the number of probe and target complexes which show a single mismatch (and therefore disintegrate) and the number of perfectly matching probe and target complexes that remain stable can be used for differentiating the two alleles.

FIG. 1 shows a graph illustrating the signal ratio Cy5/FAM of investigated alleles on a semi-logarithmic scale. It is demonstrated how the strength or weakness of the fluorescence signals of investigated alleles affect the signal ratio of the Cy5 and FAM fluorescence. For the correct interpretation of FIG. 1, the following has to be taken into consideration:
Normalization probe: A good signal means that there is target present and indicates how much. Poor signal means that there was a failure of some sort and the data is not to be trusted. Since the normalization probe binds stoichiometrically with the target, this allows quantification of the target sequence available for hybridization.
Strong allele 1 signal: If allele 2 signal is weak the target has allele 1 SNP. In FIG. 1, let the “C” SNP be allele 1 and the “G” SNP is allele 2. A very strong allele 1 probe signal (labeled with Cy5 and correlating to the “C” SNP) relative to the allele 2 probe signal (labeled with FAM and correlating to the “G” SNP) gives rise to a large ratio of the Cy5/FAM signals. This is seen with the synthetic template T39.
Weak allele 1 signal: If allele 2 signal is strong, the target has allele 2 SNP. Converse to above, template T40 gives a much higher signal with the allele 2 probe (labeled with FAM and correlating to the “G” SNP) than the allele 1 probe (labeled with Cy5 and correlating to the “C” SNP). Thus the signal ratio of Cy5/FAM is very low. The human genomic sample (CEPH 6984) also shows a similar ratio and would be determined to contain the allele 2 “G” SNP.
Roughly equal signals: Target is heterozygous for allele 1 and 2. T39/T40 in FIG. 1 represents an equimolar mixture of both synthetic probes to simulate a heterozygote (equal allele 1 and 2) and the ratio of the signals of the two probes here is about 1.
Other ratios: If the signal ratios are outside of what is characterized for these particular targets, this implies that the target is derived from a mixture of genomic DNA's. This should be also present in other SNPs in the assay as well and indicate a mixture.

The Method of Detecting SNP in Nucleic Acid Containing Samples:

When, according to the present invention, applying the method of detecting single nucleotide polymorphism (SNP) in nucleic acid containing samples, a pair of stem-loop probes according to the present invention and as already described is utilized for SNP genotyping of two individual SNP nucleic acid target sequences of a sample. In particular, a ratio of perfect match probe/target hybrids to mismatched probe/target hybrids is detected at a certain temperature and the nucleotide sequence of the stem-loop probe is chosen such that the perfect match probe/target hybrids having a melting point Tₘ that is at least 5° C. higher than the Tₘ of mismatched probe/target hybrids.

As a possible starting material, a swab head with biologic material (ideally of one single subject, e.g. a person, but may also be of many people as a mixture) is taken. The method is however not restricted to blood investigation; any biological material that yields genomic DNA connection starting material of an individual (human, animal, or plant) can be utilized. Such genomic DNA is isolated from the biological material and purified. Mitochondrial deoxyribonucleic acid (mtDNA) may also co-purify, but is usually not measured or interrogated.

When utilizing microtiter plates or the electrowetting techniques and cartridge of the co-pending U.S. patent application Ser. No. 13/188,584 (which by explicit reference is incorporated herein in its entirety), and polymerase chain reaction (PCR) for one single DNA sample, 13 different genomic targets (=13 different SNP loci) can be interrogated with 26 different stem-loop probes and 13 different normalization probes according to the invention.

However, the number of target sequences may have an influence on the PCR reaction: Since input DNA may be limiting in the instance of trace samples, it would be preferable to maximize the use of the DNA and do this as a multiplex. The maximal number would be the number of amplicons that could be produced in an optimized multiplex PCR. Sanchez et al. (“Development of a multiplex PCR assay detecting 52 autosomal SNPs” International Congress Series 2006, 1288: 67-69, which is introduced herein in its entirety) produced a 52-plex PCR reaction with 104 primers.

To ensure discrimination of certain populations, a minimum number of target sequences preferably is used in order to ensure a specific, desired conclusion as the discrimination power of the assay increases with the number of SNPs. The random match probability (R) is given by the equation:

R=(p)ⁿ [1],

where p is the individual match probability for a given SNP and n is the number of SNPs. In a simple example, if the population has 50% allele 1 and 50% allele 2 (which is the best case scenario for biallelic SNPs), then p=0.375 (the possibilities are AA, 2× AB, BB). The discrimination power of the assay is the inverse of the random match probability R, so that with 1 SNP, p=0.375 and the assay could generally discriminate an individual in a population of 3 (which is not so good). With 13 SNPs however, the assay could generally discriminate 1 in 345,000, with 21 SNPs, 1 in 881,745,755, etc. This analysis is described by D. A. Jones (“Blood samples: Probability of Discrimination” J. Forensic Sci. Soc. 1972: 355-359 included, which is introduced herein in its entirety). Here, the inventors choose 13 SNPs as a compromise to work with as few as possible for the engineering of the device as disclosed in the co-pending U.S. patent application Ser. No. 13/188,584, but to have enough to be meaningful. The SNPs of the present patent application are chosen from the paper of Sanchez et al. (see above). Other applications would probably use different SNPs.

With respect to an influence of the type of SNPs used for the analysis, the SNPs preferably are chosen such that they are conservative, non-coding, and best if they are as close as possible to 50% each allele in the populations being screened. Of course there could be other uses for stem-loop probes according to the invention, such as eye color determination of an unknown individual. There would be used SNPs shown to determine eye color. However, it is preferred following the general population rules.

It is preferred to use a 2 step PCR thermal cycling protocol to reduce the time of the assay. The temperatures are determined empirically with the particular amplicons used. More specifically, a PCR protocol was selected that gives robust and specific amplifications of all the PCR products that are to be interrogated in subsequent steps. The steps for optimizing this protocol are the same as they are for forming any multiplex PCR assay, with important factors being: having similar melting temperatures for all the PCR primers in the reaction so that they all work at a single annealing temperature. This is accomplished by adjusting the length and position of the PCR primers so that their calculated and demonstrated melting temperatures are similar. This can also include changing the position of the PCR primers if it is shown that they bind non-specifically (i.e. anneal at multiple places in the genome). There are many software programs that can facilitate this task. The empirical optimization involves conducting PCR at various temperatures regimens to show that PCR is specific and efficient around the chosen profile. The use of conventional 3-temperature PCR would also be possible.

Minimum size of an amplicon would be the total of the length of the 2 PCR primers, enough sequence to allow for the normalization probe (say 20 bp) and the hybridization region of the SNP probes (say another 20). The inventors strived to keep the PCR amplicon size to a minimum so that the complexity of the hybridization target (i.e. the PCR amplicon) is as low as possible: longer targets afford more opportunities for unwanted partial hybridization or possible secondary structure that would lower signal and increase noise.

Whether the sense primer or the antisense primer is Biotin linked in order to isolate one single strand of the two alleles while removing the other, complementary strand is completely arbitrary and is influenced by which strand of the product (sense or antisense) performs better as a SNP assay template. One of the strands may exhibit inhibitory secondary structure that the other does not. The appropriate selection usually is determined empirically. More specifically, one of the two strands may have secondary structure when rendered single stranded which would compete with probe binding. This is seen as low hybridization efficiency and low signal. Further, complementary probes may bind non-specifically to one of the two strands, which is seen as high background in a “mismatch” primer hybridization experiment. Computer programs help to predict these, but they are proven empirically.

After unbound probe material is washed away, what is detected is the fluorescence (proportional to the amount) of each of the e.g. three probes labeled with 3 different fluorophors. A normalization probe should bind stoichiometrically with the target such that amount of the normalization probe indicates the amount of the target. The allele specific probes are in competition for the same binding site surrounding the SNP with the match probe being favored over the mismatch probe.

Exemplary Hybridization Experiments:

In a first series of exemplary hybridization experiments (see FIG. 2), a discrimination ratio of greater than 4 was detected at 47° C., at 49° C., and at 51° C. The discrimination ratio is defined as the signal of perfect match probe/target hybrid divided by the signal of mismatched probe/target hybrid for the same probe:

FIG. 2A shows the result of a first experiment, revealing the A-T perfect match signal of a first probe. FIG. 2B shows the result of a second experiment, revealing the C-T mismatch signal of the same first probe. The discrimination here is 500/50=10. FIG. 2C shows the result of a third experiment, revealing the A-G mismatch signal of a second probe. FIG. 2D shows the result of a fourth experiment, revealing the C-G perfect match signal of the same second probe. The discrimination here is 300/60=5.

It is important to note here that there are two necessary discrimination measures per SNP, i.e. the results of four experiment and two probes.

In a second series of exemplary hybridization experiments (see FIG. 3), the specificity is defined as the signal of perfect match probe/target hybrid divided by the signal of mismatched probe/target hybrid for the same target:

FIG. 3A shows the result of a first experiment, revealing the A-T perfect match signal of a first probe with a first target. FIG. 3B shows the result of a second experiment, revealing the A-G mismatch signal of a second probe with the same first target. The specificity here is 600/100=6. FIG. 3C shows the result of a third experiment, revealing the C-T mismatch signal of the first probe with a second target. FIG. 3D shows the result of a fourth experiment, revealing the C-G perfect match signal of the second probe with the same second target. The specificity here is 800/100=8.

It is important to note here that there are two necessary specificity measures per SNP, i.e. the results of four experiment and two targets.

The specificity was found to be greater than 3.0 or smaller than 0.3 at 47° C., at 49° C., and at 51° C., wherein specificity is defined here as the signal of perfect match hybrid divided by the signal of mismatched hybrid for the same target.

In a third series of exemplary hybridization experiments (see FIG. 4), the resolving power is defined as the fold change in FAM/Cy5 or Cy5/FAM signal between a homozygous target (alleles A or B, see FIG. 4A) and a heterozygous target (alleles A+B, see FIG. 4B):

FIG. 4A shows the result of a first experiment, revealing the A-T perfect match signal of a first probe with a first target and the A-G mismatch signal of a second probe with the same first target. The fold change here is 500/50=10. FIG. 4B shows the result of a second experiment, revealing the A-T perfect match signal of the first probe with the first target and the C-G perfect match signal of the second probe with the second target. The fold change here is 400/200=2. The resulting resolving power₁ is (500/50)/(400/200)=10/2=5.

FIG. 4B again shows the result of the second experiment; the fold change still being is 400/200=2. FIG. 4C sows the result of a third experiment revealing the C-T mismatch signal of the first probe with the second target and the C-G perfect match signal of the second probe with the second target. The fold change here is 600/120=5. The resulting resolving power₂ is (600/120)/(200/400)=5/0.5=10.

The resolving power generally was found to be greater or equal 2.5 for paired Cy5/FAM probes. For many paired Cy5/FAM probes, the resolving power was even greater than or equal to 3.5. It is important to note here that the relative signal between FAM and Cy5 is arbitrary (because the gain on each channel is arbitrary), but as the resolving power has double ratio, arbitrariness is removed. It is important to note that (as shown) there are four Resolving Power metrics per SNP.

Probe Design:

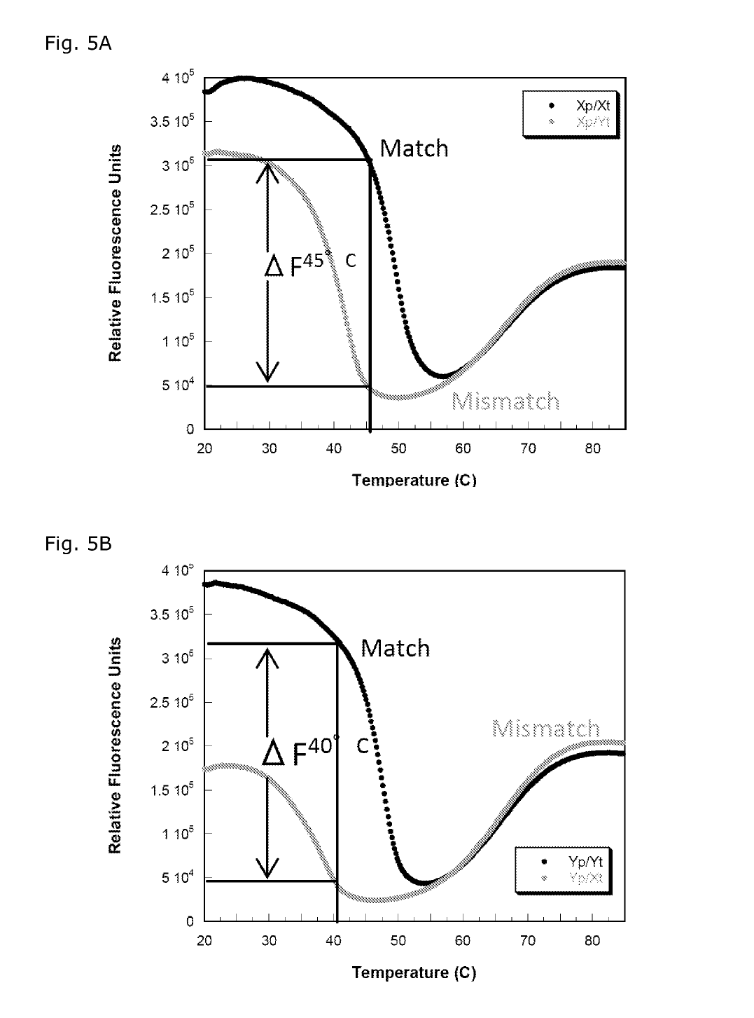

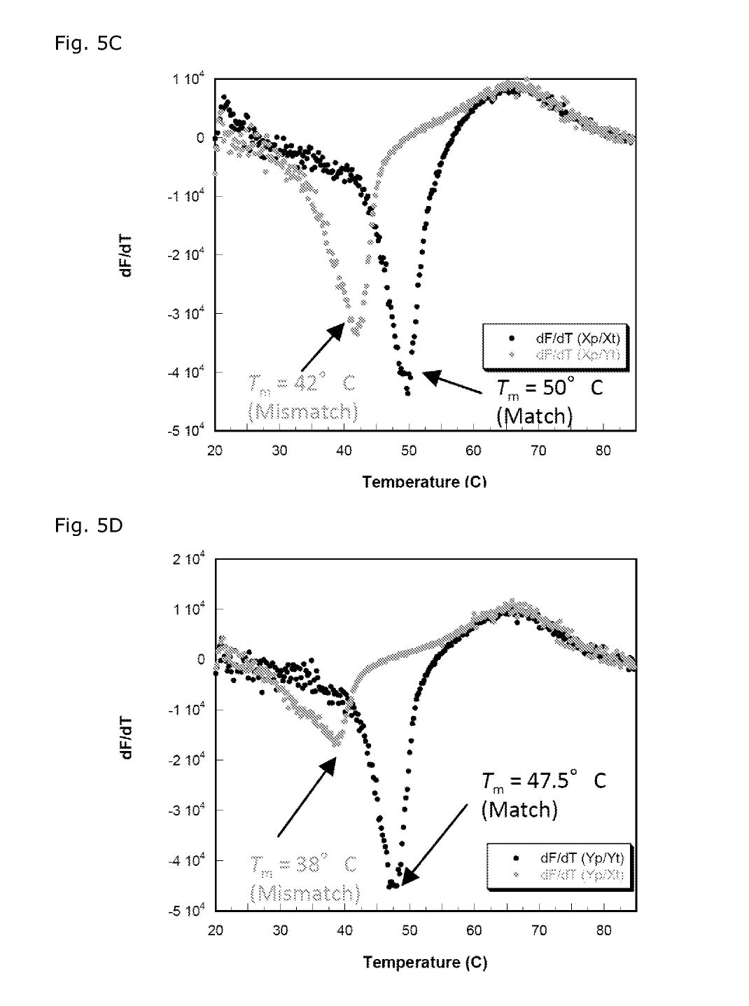

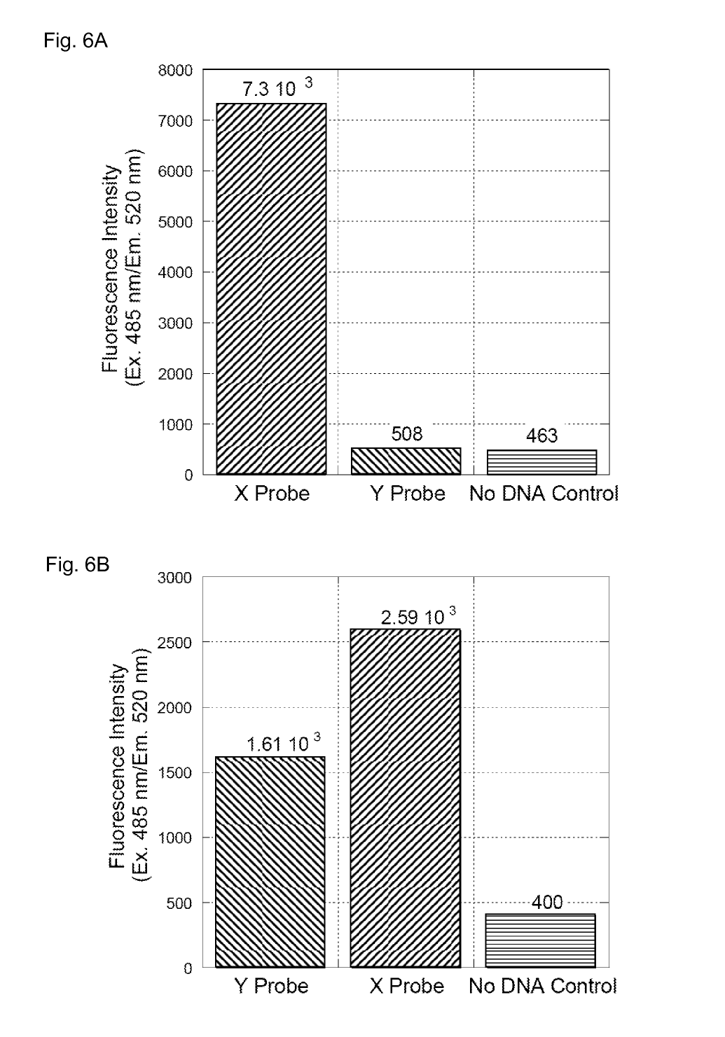

The FIG. 5 shows discrimination of Amelogenin Intron 1 X and Y alleles and demonstrates use of Tₘ data to design probes. The FIGS. 5A and 5B show the difference in Tₘs with 2 different probes designed against a C/T SNP in the Annelogenin gene Intron 1. This gene is present on both the X and Y chromosomes, but has different SNP alleles (C or T) for the X and Y chromosome and can thus be used to determine gender of a DNA contributor. For this experiment, each probe is labeled with a 5′ FAM dye and a 3′ quencher. This probe arrangement is known as a “sunrise probe” such that while the probe is a stem-loop and not hybridized to the target, the dye and quencher are held in close proximity to each other (fluorescence is low). When the probe hybridizes to its target, the dye and quencher are physically distant (fluorescence is high). This is not the preferred embodiment of the present invention, but allows rapid data gathering for this particular demonstration. For this experiment, the target sequences are interrogated individually (with one or the other allele-specific probes, not simultaneously as in the preferred embodiment). The FIGS. 5A and 5C represent interrogation of X and Y targets with an X chromosome-specific probe. The FIGS. 5B and 5D represent interrogation of X and Y targets with a Y chromosome-specific probe. The FIGS. 5A and 5B show the fluorescence signal as a function of temperature with the matched target in black and the mismatched target in grey. These are the melting curves of the probe/target pairs. From this data, one can see that the temperature at which this probe/target pair shows the greatest fluorescence difference (6-fold difference in X-probe fluorescence) between matched and mismatched probes is about 45° C. for the X allele (see indication of ΔF in FIG. 5A). From this data, one can also see that the temperature at which this probe/target pair shows the greatest fluorescence difference (6.5-fold difference in Y-probe fluorescence) between matched and mismatched probes is about 40° C. for the Y allele (see indication of ΔF in FIG. 5B).

The FIGS. 5C and 5D show the first derivatives of the fluorescence signals in the FIGS. 5A and 5B, which is indicated in both cases as in relative fluorescence units. These first derivatives help to identify the inflection points of the respective fluorescence signals (the Tₘs of each probe/target pair). This data is used to select a temperature at which both probe pairs show sufficient discrimination (signal difference) so that these probes could be used in a simultaneous experiment, as shown in FIG. 6. Alternately, this data would guide the researcher practicing the present invention to design a higher Tₘ probe for the Y-specific probe (or lower Tₘ probe for the X-specific probe) such that the two probes had more similar Tₘ.

The following Table 1 presents a selection of stem-loop probes and the respectively measured melting temperatures. These melting temperatures refer in each case to a 0.4 μM probe in HEPES buffer with 50 mM NaCl and 1 mM MgCl₂. In the Table 1, the SEQ ID: NOs of the stem-loop probes, the probe identity numbers (probe ID), the probe sequences with the attached fluorophors, and the respectively measured melting temperatures (Tₘ) are indicated. As a key for reading the probe ID, a second number designates the SNP, a second letter indicates whether the probe has a forward or reverse sequence, and a third letter indicates the identity of the SNP (underlined in probe sequence).

TABLE 1

| SEQ | | | | | |
|---|---|---|---|---|---|
| ID: | | | Tₘ | Tₘ | Tₘ |
| NO | Probe ID* | 5′ → Probe Sequence → 3′ | (PM) | (MM) | (HP) |
| 7 | P3-7RG | GCGAC-CAACGAGCGTCTTGTAA-GTCGC-FAM | 54.9 | 48.7 | 54.4 |
| 115 | P110-7RA | GCGTG-GGTCAACGAGCATCTTG-CACGC-Q670 | 56.7 | 51 | 56.8 |
| 81 | P77-9FC | GCGTG-GTTTTATTCCTGTCCCAGT-CACGC-Cy5 | 56.4 | 48.3 | 55.9 |
| 56 | P52-9FG | GCGTG-GTTTTATTGCTGTCCCAGT-CACGC-FAM | 55.1 | 49.8 | 55.9 |
| 55 | P51-9FC | GCGTG-GTTTTATTCCTGTCCCAGT-CACGC-FAM | 56.4 | 48.3 | 55.9 |
| 118 | P114-9FG | GCGTG-GTTTTATTGCTGTCCCAGT-CACGC-Cy5 | 55.1 | 49.8 | 55.9 |
| 11 | P7-14FA | GCGTG-TGAGCTGCATGTTGTTT-CACGC-FAM | 57.6 | 52.1 | 55.3 |
| 75 | P71-14FT | GCGTG-TGAGCTGCTTGTTGTTT-CACGC-Cy5 | 57.5 | 51.9 | 55.3 |
| 40 | P36-24FG | GACGC-GCCGGAGATGAGTTAGA-GCGTC-FAM | 56.6 | 50 | 55.4 |
| 76 | P72-24FA | GACGC-GCCGGAGATAAGTTAGA-GCGTC-Cy5 | 53.9 | 48 | 55.4 |
| 15 | P11-25RG | GCGTG-CATTACAAGGGGCAGC-CACGC-FAM | 56.3 | 49.1 | 55.6 |
| 73 | P69-25RA | GACGC-CATTACAAAGGGCAGCA-GCGTC-Cy5 | 56.5 | 49.3 | 55 |
| 23 | P19-34FC | GACGC-TATGGATCAGCAAGAGT-GCGTC-FAM | 54.9 | 47.3 | 54.4 |
| 77 | P73-34FG | GACGC-TATGGATGAGCAAGAGT-GCGTC-Cy5 | 54.9 | 48.8 | 54.4 |
| 27 | P23-38FC | GACGC-TGGCATCAAAGAAGGC-GCGTC-FAM | 57.2 | 49.8 | 54.4 |
| 78 | P74-38FG | GACGC-TGGCATGAAAGAAGGC-GCGTC-Cy5 | 57.2 | 51.1 | 54.4 |
| 65 | P61-40FG | GCGAG-CTCTAAGTGCGTATTTCA-CTCGC-FAM | 53.2 | 47.1 | 54.9 |
| 116 | P112-40FA | GCGAC-TCTAAGTGCATATTTCAT-GTCGC-Q670 | 53.3 | 47.9 | 54 |
| 67 | P63-42FA | TGCGTG-GTCTAAAGAGCAAAGAAGT-CACGC-FAM | 55.3 | 49.2 | 55 |
| 85 | P81-42FG | GCGTG-TGTCTAAAGGGCAAAGAAG-CACGC-Cy5 | 56.6 | 51.3 | 56.7 |
| 92 | P88-43RG | GAGGC-CTGTCCCGTCTACTTA-GCCTC-FAM | 53.7 | 45.5 | 53.3 |
| 120 | P126-43RA | GAGGC-GCTGTCCCATCTACTTA-GCCTC-Q670 | 53.8 | 47.3 | 53 |
| 71 | P67-49FG | FAM-CGGTC-GGAATTGAGTCGCCG-GACCG | 55.6 | 48.2 | 55 |
| 117 | P113-49FA | Q670-CCGGA-CGTGGAATTAAGTCGC-TCCGG | 53.7 | 47.7 | 52.7 |
| 33 | P29-50RG | GACGC-TGCGCTACGTAACTCT-GCGTC-FAM | 56.2 | 49.6 | 55.3 |
| 74 | P70-50RA | GACGC-TGCGCTACATAACTCTT-GCGTC-Cy5 | 54.5 | 48.2 | 54.4 |
| 114 | P100-59FG | GCTCG-AGTCTCGCAGCCAC-CGAGC-FAM | 56.5 | 48.4 | 55.2 |
| 119 | P120-59FA | CGCGT-TTAGTCTCACAGCCACAT-ACGCG-Q670 | 57.1 | 49.7 | 56.5 |

In the three columns on the right of this Table 1, the following is indicated:
Tₘ (PM) is the melting temperature of that probe with the perfect match target;
Tₘ (MM) is the melting temperature of that probe with the mismatch target; and
Tₘ(HP) is the intramolecular melting temperature for the stem loop probe alone.

Genotyping Assay:

The FIG. 6 shows the data from an X/Y typing experiment using data from FIG. 5 at a single temperature. In this experiment, each probe is singly labeled (FAM in this case, no quencher probe) as is described in the preferred embodiment.

First, the probe sequences from FIG. 5 are used to interrogate a sample of DNA derived from a female contributor (XX or homozygous for the X allele, see FIG. 6A). Differential hybridization of immobilized female (7437) or male (7432) Annelogenin amplicon is displayed here. The fluorescence intensity is indicated as a result of the fluorescence emission measured at 520 nm after excitation at 485 nm. The fluorescence signal of the X probe is more than 14 times higher than the fluorescence signal of the Y probe and the signal of the control without any DNA present is about equal to the signal of the Y probe (see indicated values on the top of the respective bar-graphs). A strong signal from the X-allele specific probe and background signal from the Y-allele specific probe at 47° C. is detected here. These results clearly demonstrate the presence of the female (7437) Amelogenin amplicon.

Then, the probe sequences from FIG. 5 are used to interrogate a sample of DNA derived from a male DNA contributor (XY or heterozygous for the X and Y alleles, see FIG. 6B). Differential hybridization of immobilized male (7432) Amelogenin amplicon is displayed here. The fluorescence intensity again is indicated as a result of the fluorescence emission measured at 520 nm after excitation at 485 nm. The fluorescence signal of the Y probe is more than 4 times higher than the fluorescence signal of the control without any DNA present. The fluorescence signal of the X probe is more than 6 times higher than the fluorescence signal of the control without any DNA present (see indicated values on the top of the respective bar-graphs). Compared with the control, the signals of the Y probe and of the X probe can be regarded to be about equal. Strong signal from both allele-specific probes is detected here, again at a temperature 47° C. These results clearly demonstrate the presence of the male (7432) Amelogenin amplicon.

It is thus demonstrated that in an assay performed using 3′-FAM labeled stem-loop probes at 47° C., hybridization with stem-loop probes can successfully genotype Amelogenin.

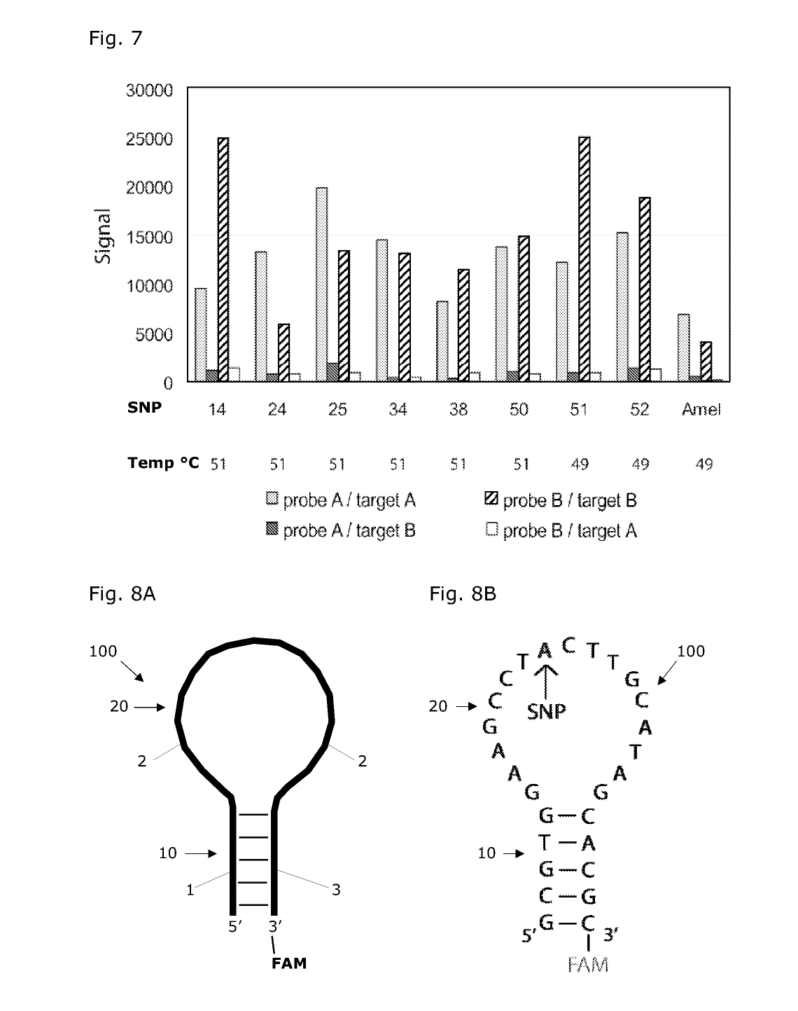

The FIG. 7 shows the data from several SNP experiments (SNPs are numbered 14, 24, 25, etc on the bottom of the graph). The graph is the signal from the hybridization experiments with 4 different configurations for each SNP (in this order):
Match probe A with target A
MISmatch probe A with target B
Match probe B with target B
MISmatch probe B with target A

As expected, the signal with the match arrangements is much higher than the signal with the mismatch arrangements (more probe binds to the match target).

Below the graph of signals is listed the temperature at which each SNP experiment was carried out. Here, each probe of the set (match and mismatch) was designed to perform at the same temperature as discussed above.

The desired outcome as displayed in this data is both very high signal with the match configurations (reaching a maximum at 1:1 binding of match probe to target) and very low signal in the mismatch configurations (reaching a minimum at zero mismatch probe binding to target) such that the ratio of match signal to mismatch signal is as high as possible.

FIG. 8 shows graphic models of stem-loop probes according to the invention. FIG. 8A shows the basic parts of a stem-loop probe 100, comprising first 1, second 2, and third 3 single stranded nucleic acid portions. The second single stranded nucleic acid portion 2 is located between the first 1 and the third 3 single stranded nucleic acid portions, the first 1 and the third 3 single stranded nucleic acid portions building a double stranded, intramolecular stem 10. The second single stranded nucleic acid portion 2 forms a single stranded oligonucleotide loop 20. FIG. 8B shows a typical sequence of a stem-loop probe 100 with a 17 nucleotide loop 20. In position 8 of the single stranded oligonucleotide loop 20, a single nucleotide polymorphism (SNP, see arrow) is indicated by one Adenine. The stem 10 of the stem-loop probe 100 is composed of 5 base pairs. Here, an FAM fluorophor is attached to the 3′ end of the nucleotide sequence of the stem-loop probe 100.

What is claimed is:

1. A stem-loop probe for single nucleotide polymorphism (SNP) genotyping of individual SNP nucleic acid target sequences; the stem-loop probe (100) comprising first (1), second (2), and third (3) single stranded nucleic acid portions; the second single stranded nucleic acid portion (2) being located between the first (1) and the third (3) single stranded nucleic acid portions; the first (1) and the third (3) single stranded nucleic acid portions building a double stranded, intramolecular stem (10); and the second single stranded nucleic acid portion (2) forming a single stranded oligonucleotide loop (20) with a nucleotide sequence that is complementary to individual SNP nucleic acid target sequences,
wherein the nucleotide sequence of the stem-loop probe is chosen such that perfect match probe/target hybrids have a melting point Tₘ that is at least 5° C. higher than the Tₘ of mismatched probe/target hybrids.

2. The stem-loop probe of claim 1,
wherein the intramolecular stem (10) of the probes and a perfect match probe/target hybrid have a Tₘ of close to 55° C. in HEPES buffer with 50 mM NaCl and 1 mM MgCl₂ in the presence of 0.4 μM probe concentration.

3. The stem-loop probe of claim 1,
wherein the intramolecular stem (10) is composed of 5 base pairs.

4. The stem-loop probe of claim 3,
wherein the five base pairs of the intramolecular stem (10) comprise 4 G-C base pairs.

5. The stem-loop probe of claim 1,
wherein the single stranded oligonucleotide loop (20) is free of secondary structure.

6. The stem-loop probe of claim 1,
wherein the single stranded oligonucleotide loop (20) has a length of 15 to 18 nucleotides.

7. The stem-loop probe of claim 1,
wherein the first single stranded nucleic acid portion (1) has the nucleotide sequence of SEC ID: NO 1, and
wherein the third single stranded nucleic acid portion (3) has the nucleotide sequence of SEC ID: NO 3.

8. The stem-loop probe of claim 1,
wherein the single stranded oligonucleotide loop (20) has the nucleotide sequence of SEC ID: NO 2 or of SEC ID: NO 4.

9. The stem-loop probe of claim 7,
wherein the first (1) and the third (3) single stranded nucleic acid portions comprise a 3′ or 5′ end configured as an A, T, or C nucleotide to which A, T, or C nucleotide a fluorophore is conjugated.

10. The stem-loop probe of claim 9,
wherein the fluorophore is FAM or Cy5 or Q670.

11. The stem-loop probe of claim 7,
wherein the single stranded oligonucleotide loop (20) has the nucleotide sequence of SEC ID: NO 2 or of SEC ID: NO 4, and
wherein the fluorophore is FAM or Cy5 conjugated to the 3′ end of the nucleotide sequence of SEC ID : NO 3.

12. A pair of loop probes for single nucleotide polymorphism (SNP) genotyping of two individual SNP nucleic acid target sequences in a single sample according to claim 1,
wherein a first loop probe has the nucleotide sequence of SEC ID: NO 5 with a FAM fluorophore conjugated to the 3′ end, and
wherein a second loop probe has the nucleotide sequence of SEC ID: NO 6 with a Cy5 or Q670 fluorophore conjugated to the 3′ end.

13. A method of detecting single nucleotide polymorphism (SNP) in nucleic acid containing samples, utilizing a pair of stem-loop probes for SNP genotyping of two individual SNP nucleic acid target sequences of a sample; the stem-loop probes (100) comprising first (3), second (2), and third (3) single stranded nucleic acid portions; the second (2) single stranded nucleic acid portion being located between the first (1) and the third (3) single stranded nucleic acid portions; the first (1) and the third (3) single stranded nucleic acid portions building a double stranded, intramolecular stem (10); and the second (2) single stranded nucleic acid portion forming a single stranded oligonucleotide loop (20) with a nucleotide sequence that is complementary to one of the individual SNP nucleic acid target sequences,
wherein a ratio of perfect match probe/target hybrids to mismatched probe/target hybrids is detected at a certain temperature, and wherein the nucleotide sequence of the stem-loop probe is chosen such that the perfect match probe/target hybrids having a melting point Tₘ that is at least 5° C. higher than the Tₘ of mismatched probe/target hybrids.

14. The method of claim 13,
wherein the intramolecular stem (10) is composed of 5 base pairs.

15. The method of claim 14,
wherein the five base pairs of the intramolecular stem (10) comprise 4 G-C base pairs.

16. The method of claim 13,
wherein the single stranded oligonucleotide loop (20) is free of secondary structure.

17. The method of claim 13,
wherein the single stranded oligonucleotide loop (20) has a length of 15 to 18 nucleotides.

18. The method of claim 13,
wherein the first single stranded nucleic acid portion (1) has the nucleotide sequence of SEC ID: NO 1, and
wherein the third single stranded nucleic acid portion (3) has the nucleotide sequence of SEC ID: NO 3.

19. The method of claim 13,
wherein the single stranded oligonucleotide loop (20) has the nucleotide sequence of SEC ID: NO 2 or of SEC ID: NO 4.

20. The method of claim 18,
wherein the first (1) and the third (3) single stranded nucleic acid portions comprise a 3′ or 5′ end configured as an A, T, or C nucleotide to which A, T, or C nucleotide a fluorophore is conjugated.

21. The method of claim 20,
wherein the fluorophore is FAM or Cy5 or Q670.

22. The method of claim 18,
wherein the single stranded oligonucleotide loop (20) has the nucleotide sequence of SEC ID: NO 2 or of SEC ID: NO 4, and
wherein the fluorophore is FAM or Cy5 or Q670 conjugated to the 3′ end of the nucleotide sequence of SEC ID: NO 3.

23. The method of claim 13,
wherein is utilized a pair of loop probes for single nucleotide polymorphism (SNP) genotyping of two individual SNP nucleic acid target sequences in a single sample,
wherein a first loop probe has the nucleotide sequence of SEC ID: NO 5 with a FAM fluorophore conjugated to the 3′ end, and
wherein a second loop probe has the nucleotide sequence of SEC ID: NO 6 with a Cy5 or Q670 fluorophore conjugated to the 3′ end.

24. The method of claim 13,
wherein detection of the ratio of perfect match probe/target hybrids to mismatched probe/target hybrids is carried out at about 55° C. in HEPES buffer with 50 mM NaCl and 1 mM MgCl₂ in the presence of 0.4 μM probe concentration.


SEQUENCE LISTING (122 sequences)

SEQ ID NO: 1 - DNA, 5 bp, Artificial Sequence
  Synthetic
gcgtg

SEQ ID NO: 2 - DNA, 19 bp, Artificial Sequence
  Synthetic
gttttattgctgtcccagt

SEQ ID NO: 3 - DNA, 5 bp, Artificial Sequence
  Synthetic
cacgc

SEQ ID NO: 4 - DNA, 19 bp, Artificial Sequence
  Synthetic
gttttattcctgtcccagt

SEQ ID NO: 5 - DNA, 27 bp, Artificial Sequence
  Synthetic
gcgtgtctttgcaaaactggctcacgc

SEQ ID NO: 6 - DNA, 27 bp, Artificial Sequence
  Synthetic
gcgtgtctttgcagaactggctcacgc

SEQ ID NO: 7 - DNA, 27 bp, Artificial Sequence
  Synthetic
gcgaccaacgagcgtcttgtaagtcgc

SEQ ID NO: 8 - DNA, 27 bp, Artificial Sequence
  Synthetic
gcgaccaacgagcatcttgtaagtcgc

SEQ ID NO: 9 - DNA, 24 bp, Artificial Sequence
  Synthetic
gcctccctgctgacttcccgaggc

SEQ ID NO: 10 - DNA, 23 bp, Artificial Sequence
  Synthetic
gcctcctgctggcttcccgaggc

SEQ ID NO: 11 - DNA, 27 bp, Artificial Sequence
  Synthetic
gcgtgtgagctgcatgttgtttcacgc

SEQ ID NO: 12 - DNA, 27 bp, Artificial Sequence
  Synthetic
gcgtgtgagctgcttgttgtttcacgc

SEQ ID NO: 13 - DNA, 27 bp, Artificial Sequence
  Synthetic
gccctccggagatgagttagaaagggc

SEQ ID NO: 14 - DNA, 27 bp, Artificial Sequence
  Synthetic
gccctgccggagataagttagaagggc

SEQ ID NO: 15 - DNA, 26 bp, Artificial Sequence
  Synthetic
gcgtgcattacaaggggcagccacgc

SEQ ID NO: 16 - DNA, 27 bp, Artificial Sequence
  Synthetic
gacgccattacaaagggcagcagcgtc

SEQ ID NO: 17 - DNA, 26 bp, Artificial Sequence
  Synthetic
gcgaccatccttcaagctccagtcgc

SEQ ID NO: 18 - DNA, 25 bp, Artificial Sequence
  Synthetic
gcctgatccttcgagctccacaggc

SEQ ID NO: 19 - DNA, 27 bp, Artificial Sequence
  Synthetic
gcgacagcatgggacatttcacgtcgc

SEQ ID NO: 20 - DNA, 25 bp, Artificial Sequence
  Synthetic
ggtgggcatggggcatttcaccacc

SEQ ID NO: 21 - DNA, 26 bp, Artificial Sequence
  Synthetic
gcgtcctccaaatgcggtcacgacgc

SEQ ID NO: 22 - DNA, 27 bp, Artificial Sequence
  Synthetic
gcccgctccaaatacggtcacacgggc

SEQ ID NO: 23 - DNA, 27 bp, Artificial Sequence
  Synthetic
gacgctatggatcagcaagagtgcgtc

SEQ ID NO: 24 - DNA, 27 bp, Artificial Sequence
  Synthetic
gacgctatggatgagcaagagtgcgtc

SEQ ID NO: 25 - DNA, 25 bp, Artificial Sequence
  Synthetic
ggccctagccacatgcagccgggcc

SEQ ID NO: 26 - DNA, 23 bp, Artificial Sequence
  Synthetic
ggcccagccacgtgcagcgggcc

SEQ ID NO: 27 - DNA, 26 bp, Artificial Sequence
  Synthetic
gacgctggcatcaaagaaggcgcgtc

SEQ ID NO: 28 - DNA, 26 bp, Artificial Sequence
  Synthetic
gacgctggcatgaaagaaggcgcgtc

SEQ ID NO: 29 - DNA, 26 bp, Artificial Sequence
  Synthetic
gcgacgggatgaaggtggagagtcgc

SEQ ID NO: 30 - DNA, 25 bp, Artificial Sequence
  Synthetic
gcgacgggatgagggtggaggtcgc

SEQ ID NO: 31 - DNA, 28 bp, Artificial Sequence
  Synthetic
gcgtgggctgtcccatctacttacacgc

SEQ ID NO: 32 - DNA, 27 bp, Artificial Sequence
  Synthetic
gccctgctgtcccgtctacttaagggc

SEQ ID NO: 33 - DNA, 26 bp, Artificial Sequence
  Synthetic
gacgctgcgctacgtaactctgcgtc

SEQ ID NO: 34 - DNA, 27 bp, Artificial Sequence
  Synthetic
gacgctgcgctacataactcttgcgtc

SEQ ID NO: 35 - DNA, 27 bp, Artificial Sequence
  Synthetic
gcgtggaagcctacttgcatagcacgc

SEQ ID NO: 36 - DNA, 27 bp, Artificial Sequence
  Synthetic
gcgtggaagcctgcttgcatagcacgc

SEQ ID NO: 37 - DNA, 26 bp, Artificial Sequence
  Synthetic
gccctgccgtggtatactgagagggc

SEQ ID NO: 38 - DNA, 26 bp, Artificial Sequence
  Synthetic
gccctgccgtggaatactgagagggc

SEQ ID NO: 39 - DNA, 27 bp, Artificial Sequence
  Synthetic
gcgtgggtcaacgagcatcttgcacgc

SEQ ID NO: 40 - DNA, 27 bp, Artificial Sequence
  Synthetic
gacgcgccggagatgagttagagcgtc

SEQ ID NO: 41 - DNA, 27 bp, Artificial Sequence
  Synthetic
gacgcgccggagataagttagagcgtc

SEQ ID NO: 42 - DNA, 26 bp, Artificial Sequence
  Synthetic
gcctgcatccttcgagctccacaggc

SEQ ID NO: 43 - DNA, 23 bp, Artificial Sequence
  Synthetic
gacgcctgctgacttcccgcgtc

SEQ ID NO: 44 - DNA, 23 bp, Artificial Sequence
  Synthetic
accccctgctggcttcccggggt

SEQ ID NO: 45 - DNA, 26 bp, Artificial Sequence
  Synthetic
ccccacatccttcgagctccatgggg

SEQ ID NO: 46 - DNA, 27 bp, Artificial Sequence
  Synthetic
cgcgtgcatccttcgagctccaacgcg

SEQ ID NO: 47 - DNA, 28 bp, Artificial Sequence
  Synthetic
gcgacggagcatgggacatttcagtcgc

SEQ ID NO: 48 - DNA, 25 bp, Artificial Sequence
  Synthetic
gcctcgcatggggcatttcagaggc

SEQ ID NO: 49 - DNA, 27 bp, Artificial Sequence
  Synthetic
gacgcctccaaatacggtcacagcgtc

SEQ ID NO: 50 - DNA, 20 bp, Artificial Sequence
  Synthetic
agccagggatgaaggtggct

SEQ ID NO: 51 - DNA, 26 bp, Artificial Sequence
  Synthetic
gcgacaatttggggagcttgggtcgc

SEQ ID NO: 52 - DNA, 26 bp, Artificial Sequence
  Synthetic
gcgacaatttggggtgcttgggtcgc

SEQ ID NO: 53 - DNA, 26 bp, Artificial Sequence
  Synthetic
acccgaactgtgtggtgaacgcgggt

SEQ ID NO: 54 - DNA, 28 bp, Artificial Sequence
  Synthetic
agcccaactgtgtagtgaacgtcgggct

SEQ ID NO: 55 - DNA, 29 bp, Artificial Sequence
  Synthetic
gcgtggttttattcctgtcccagtcacgc

SEQ ID NO: 56 - DNA, 29 bp, Artificial Sequence
  Synthetic
gcgtggttttattgctgtcccagtcacgc

SEQ ID NO: 57 - DNA, 26 bp, Artificial Sequence
  Synthetic
gccgacattcagctagcacattcggc

SEQ ID NO: 58 - DNA, 25 bp, Artificial Sequence
  Synthetic
gggtgcattcagctggcacacaccc

SEQ ID NO: 59 - DNA, 25 bp, Artificial Sequence
  Synthetic
agcgcttggaaagtttgcctgcgct

SEQ ID NO: 60 - DNA, 28 bp, Artificial Sequence
  Synthetic
gcgtgacttggaaaatttgcctgcacgc

SEQ ID NO: 61 - DNA, 26 bp, Artificial Sequence
  Synthetic
ggcacactttcaagtccctgcgtgcc

SEQ ID NO: 62 - DNA, 27 bp, Artificial Sequence
  Synthetic
ggcacactttcaattccctgcagtgcc

SEQ ID NO: 63 - DNA, 28 bp, Artificial Sequence
  Synthetic
gcgacgattgctcatgcacttaagtcgc

SEQ ID NO: 64 - DNA, 29 bp, Artificial Sequence
  Synthetic
gcgacggattgctcatacacttaagtcgc

SEQ ID NO: 65 - DNA, 28 bp, Artificial Sequence
  Synthetic
gcgagctctaagtgcgtatttcactcgc

SEQ ID NO: 66 - DNA, 28 bp, Artificial Sequence
  Synthetic
gcgactctaagtgcatatttcatgtcgc

SEQ ID NO: 67 - DNA, 30 bp, Artificial Sequence
  Synthetic
tgcgtggtctaaagagcaaagaagtcacgc

SEQ ID NO: 68 - DNA, 29 bp, Artificial Sequence
  Synthetic
gcgtgtgtctaaagggcaaagaagcacgc

SEQ ID NO: 69 - DNA, 22 bp, Artificial Sequence
  Synthetic
gaccgagagggcgacaccggtc

SEQ ID NO: 70 - DNA, 25 bp, Artificial Sequence
  Synthetic
ccggaaaagagggcaacacctccgg

SEQ ID NO: 71 - DNA, 25 bp, Artificial Sequence
  Synthetic
cggtcggaattgagtcgccggaccg

SEQ ID NO: 72 - DNA, 26 bp, Artificial Sequence
  Synthetic
gccctgaattaagtcgccgctagggc

SEQ ID NO: 73 - DNA, 27 bp, Artificial Sequence
  Synthetic
gacgccattacaaagggcagcagcgtc

SEQ ID NO: 74 - DNA, 27 bp, Artificial Sequence
  Synthetic
gacgctgcgctacataactcttgcgtc

SEQ ID NO: 75 - DNA, 27 bp, Artificial Sequence
  Synthetic
gcgtgtgagctgcttgttgtttcacgc

SEQ ID NO: 76 - DNA, 27 bp, Artificial Sequence
  Synthetic
gacgcgccggagataagttagagcgtc

SEQ ID NO: 77 - DNA, 27 bp, Artificial Sequence
  Synthetic
gacgctatggatgagcaagagtgcgtc

SEQ ID NO: 78 - DNA, 26 bp, Artificial Sequence
  Synthetic
gacgctggcatgaaagaaggcgcgtc

SEQ ID NO: 79 - DNA, 27 bp, Artificial Sequence
  Synthetic
gcgtggaagcctgcttgcatagcacgc

SEQ ID NO: 80 - DNA, 26 bp, Artificial Sequence
  Synthetic
gccctgccgtggaatactgagagggc

SEQ ID NO: 81 - DNA, 29 bp, Artificial Sequence
  Synthetic
gcgtggttttattcctgtcccagtcacgc

SEQ ID NO: 82 - DNA, 26 bp, Artificial Sequence
  Synthetic
gccgacattcagctagcacattcggc

SEQ ID NO: 83 - DNA, 27 bp, Artificial Sequence
  Synthetic
ggcacactttcaattccctgcagtgcc

SEQ ID NO: 84 - DNA, 28 bp, Artificial Sequence
  Synthetic
gcgtgacttggaaaatttgcctgcacgc

SEQ ID NO: 85 - DNA, 29 bp, Artificial Sequence
  Synthetic
gcgtgtgtctaaagggcaaagaagcacgc

SEQ ID NO: 86 - DNA, 27 bp, Artificial Sequence
  Synthetic
gcgaccaacgagcgtcttgtaagtcgc

SEQ ID NO: 87 - DNA, 24 bp, Artificial Sequence
  Synthetic
gccctcctgctggcttcccagggc

SEQ ID NO: 88 - DNA, 23 bp, Artificial Sequence
  Synthetic
gccctctgctggcttcccagggc

SEQ ID NO: 89 - DNA, 27 bp, Artificial Sequence
  Synthetic
gcgacattgctcatgcacttaagtcgc

SEQ ID NO: 90 - DNA, 26 bp, Artificial Sequence
  Synthetic
gacgcctccaaatacggtcacgcgtc

SEQ ID NO: 91 - DNA, 27 bp, Artificial Sequence
  Synthetic
gaggcgctgtcccatctacttagcctc

SEQ ID NO: 92 - DNA, 26 bp, Artificial Sequence
  Synthetic
gaggcctgtcccgtctacttagcctc

SEQ ID NO: 93 - DNA, 26 bp, Artificial Sequence
  Synthetic
ccggacgtggaattaagtcgctccgg

SEQ ID NO: 94 - DNA, 24 bp, Artificial Sequence
  Synthetic
gcctcgcctgctgctctgagaggc

SEQ ID NO: 95 - DNA, 24 bp, Artificial Sequence
  Synthetic
gcctcgcctgctcctctgagaggc

SEQ ID NO: 96 - DNA, 27 bp, Artificial Sequence
  Synthetic
gcgtgataggccagtttcatcccacgc

SEQ ID NO: 97 - DNA, 28 bp, Artificial Sequence
  Synthetic
gcgaccataggcaagtttcatccgtcgc

SEQ ID NO: 98 - DNA, 28 bp, Artificial Sequence
  Synthetic
gcgtgaagcaatagggtcaagagcacgc

SEQ ID NO: 99 - DNA, 30 bp, Artificial Sequence
  Synthetic
gcgtgaagcaatagagtcaagagtacacgc

SEQ ID NO: 100 - DNA, 29 bp, Artificial Sequence
  Synthetic
gcagccagagtatccagagttagcgctgc

SEQ ID NO: 101 - DNA, 27 bp, Artificial Sequence
  Synthetic
gcgacagtatccggagttagccgtcgc

SEQ ID NO: 102 - DNA, 26 bp, Artificial Sequence
  Synthetic
gcgtgatccctcgaggaacagcacgc

SEQ ID NO: 103 - DNA, 27 bp, Artificial Sequence
  Synthetic
gcgtgaatccctcaaggaacagcacgc

SEQ ID NO: 104 - DNA, 24 bp, Artificial Sequence
  Synthetic
gctcgagtctcgcagccaccgagc

SEQ ID NO: 105 - DNA, 28 bp, Artificial Sequence
  Synthetic
cgcgtttagtctcacagccacatacgcg

SEQ ID NO: 106 - DNA, 26 bp, Artificial Sequence
  Synthetic
ccggactttatgggttgcccttccgg

SEQ ID NO: 107 - DNA, 27 bp, Artificial Sequence
  Synthetic
gccctgctttatggattgccctagggc

SEQ ID NO: 108 - DNA, 26 bp, Artificial Sequence
  Synthetic
gaggcccacacatacagctcagcctc

SEQ ID NO: 109 - DNA, 24 bp, Artificial Sequence
  Synthetic
cctccggccacacgtacagggagg

SEQ ID NO: 110 - DNA, 23 bp, Artificial Sequence
  Synthetic
ctgcgcctctgggcagcccgcag

SEQ ID NO: 111 - DNA, 24 bp, Artificial Sequence
  Synthetic
gcgacccctctgagcagccgtcgc

SEQ ID NO: 112 - DNA, 29 bp, Artificial Sequence
  Synthetic
gcgtgatttttgcatcaccttttccacgc

SEQ ID NO: 113 - DNA, 27 bp, Artificial Sequence
  Synthetic
gcgtgttttgcgtcaccttttccacgc

SEQ ID NO: 114 - DNA, 24 bp, Artificial Sequence
  Synthetic
gctcgagtctcgcagccaccgagc

SEQ ID NO: 115 - DNA, 27 bp, Artificial Sequence
  Synthetic
gcgtgggtcaacgagcatcttgcacgc

SEQ ID NO: 116 - DNA, 28 bp, Artificial Sequence
  Synthetic
gcgactctaagtgcatatttcatgtcgc

SEQ ID NO: 117 - DNA, 26 bp, Artificial Sequence
  Synthetic
ccggacgtggaattaagtcgctccgg

SEQ ID NO: 118 - DNA, 29 bp, Artificial Sequence
  Synthetic
gcgtggttttattgctgtcccagtcacgc

SEQ ID NO: 119 - DNA, 28 bp, Artificial Sequence
  Synthetic
cgcgtttagtctcacagccacatacgcg

SEQ ID NO: 120 - DNA, 27 bp, Artificial Sequence
  Synthetic
gaggcgctgtcccatctacttagcctc

SEQ ID NO: 121 - DNA, 51 bp, Artificial Sequence
  Synthetic
tgcttatgaaaaatcactgggacaggaataaaacacctgggttcttttcct

SEQ ID NO: 122 - DNA, 51 bp, Artificial Sequence
  Synthetic
tgcttatgaaaaatcactgggacagcaataaaacacctgggttcttttcct